(19) United States
(12) Patent Application Publication
(10) Pub. No.: US 2024/0108274 A1
(43) Pub. Date: Apr. 4, 2024

(54) Control Signal Viewer For Prosthetic Devices

(71) Applicant: INFINITE BIOMEDICAL TECHNOLOGIES, LLC, Baltimore, MD (US)

(72) Inventors: György Miklós Lévay, Budapest (HU); Dániel Czeiner, Budapest (HU); Rahul Reddy Kaliki, Baltimore, MD (US)

(21) Appl. No.: 18/476,093

(22) Filed: Sep. 27, 2023

Related U.S. Application Data
(60) Provisional application No. 63/412,267, filed on Sep. 30, 2022.

(51) Int. Cl. A61B 5/389; A61B 5/00

(52) U.S. Cl. CPC A61B 5/389; A61B 5/7267; A61B 5/743; A61B 5/7435

(57) ABSTRACT

Embodiments are directed to a human-machine interface including one or more sensors configured to detect myoelectrical signals of a user and an electronic device configured to provide a set of training data based on the myoelectric signals as input to a classifier. The electronic device can receive one or more sets of feature data each associated with movement classification from the classification model and display a user interface including a multi-dimensional plot comprising at least two dimensions that are each based on a component dimension determined by the classifier and used to determine the first and second sets of the feature data. The plot can include visual elements corresponding to the sets of feature data. The user device can receive a second set of detected myoelectric signals and display a graphical icon on the multi-dimensional plot indicating a current feature based on the second set of myoelectric signals.

### CROSS-REFERENCE TO RELATED APPLICATION

This application is a non-provisional application and claims the benefit of U.S. Provisional Patent Application No. 63/412,267, filed Sep. 30, 2022, the disclosure of which is hereby incorporated by reference herein in its entirety.

### FIELD

The described embodiments relate generally to control systems for user-worn electromechanical prosthetic devices and in particular to adaptive control systems for prosthetic devices.

### BACKGROUND

Prosthetic devices can be used by amputee users to restore partial or complete limb function. A myoelectric prosthetic device can leverage electromyography to receive and interpret electrical signals, detected from electrodes positioned over a user's skeletal musculature, as positioning or pose instructions that, in turn, can be used to drive one or more electromechanical actuators of the prosthetic device.

A prosthetic device may be calibrated by instructing a user to perform a specific pose or movement and correlating collected myoelectric data to the pose performed by the user. This can be repeated for multiple poses or movements. However, in some cases, the myoelectric signals for different poses may be similar, which can lead to misclassification of a user's intent and cause the prosthetic to perform an unintended movement, or to transition to an unintended pose.

### BRIEF DESCRIPTION OF THE DRAWINGS

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

In particular, the disclosure will be readily understood by the following detailed description in conjunction with the accompanying drawings, wherein like reference numerals designate like structural elements, and in which:

FIG. 1 depicts an example prosthetic device that can be used with the control signal viewer as described herein;

FIG. 2 depicts an example system diagram of a control system of a myoelectric prosthetic device as described herein;

FIG. 3 depicts an example process for generating feature data that can be displayed by the control signal viewer as described herein;

FIGS. 4A-4B depict example user interfaces of a control signal viewer that display feature data for a user of a prosthetic device as described herein;

FIG. 5 depicts a process for updating a calibrated movement or creating a new movement based on feature data collected using the control signal viewer;

FIGS. 6A-6C depict example user interfaces of a control signal viewer that can be used to visualize and obtain additional feature data for a user; and

FIG. 7 depicts an example system diagram of an electronic device that may perform operations as described herein.

It should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Embodiments described herein are directed to a control signal viewer that can be used to view calibrated feature data that is associated with one or more movement classifications and used to control prosthetic devices.

The systems and methods described herein leverage classification models to identify an intended user (also may be referred to as a “patient” or “wearer” or “operator” of a prosthetic device) movement from detected volitional motor control signals, which may include myoelectric signals, sonomyography signals, and/or suitable biological signals. The classification model(s) can be initially calibrated to identify an intended user movement based on a pattern of electromyograph (EMG) signals that are obtained by one or more sensors contacting a user. The classification models can include one or more sets of feature data that are each associated with a different movement classification. For example, a first set of a feature data may be associated with a first movement classification (e.g., a pinch movement) and a second set of feature data may be associated with a second movement classification (e.g., a hand flat movement).

The examples described herein are presented in the context of using EMG signals to create and/or calibrate classification models that are used to identify intended user movement. However, other types of user input can be used to create, calibrate and/or otherwise develop classification models that used to identify intended user movement. For example, sonomyography signals can be used as an alternative to or in addition to EMG signals. The sonomyography signals may be obtained by one or more sensor contacting the user and the signals can be used to classify user movements using the techniques described herein.

The feature data for a classification model can be displayed to a user as a plot or graph that shows how the classification model has separated feature data associated with different movement classifications. For example, the classification model may include discriminant analysis, such as a linear discriminant analysis (LDA), that is used to separate the feature data associated with different movement classifications. The system may collect EMG data from each different movement and generate a feature data set for each movement using the EMG data. The classification model may determine a subspace projection that separates the class data clusters while reducing the scatter of each cluster, such that class separation boundaries can be determined. During operation, an intended movement can be identified based on which boundary the feature data associated with a current user contraction falls into.

In some cases the control signal viewer can be displayed as a plot that includes representative feature data for each set of feature data and each set of feature data may indicate the associated movement classification. This can allow a user to visualize how the classifier is separating data associated with different movement classifications. The plot can be a multi-dimensional plot that plots the classification data according to the component dimensions determined by the classification model and are used to separate the different sets of feature data. For example, the plot can be a three-dimensional plot where three orthogonal axes each correspond to a different component dimension determined by the classification model. In other cases, the plot can be displayed using other dimensional schemes such as a one-dimensional plot, a two-dimensional plot and so on.

The control signal viewer can display a user's current feature data that is determined in real-time (or near-real time) from EMG sensors that are contacting a user. The control signal viewer can display the user's current data along with the calibrated feature data sets for various movement classes, which can allow a user to see how a current muscle contraction would be classified by the classifier. As a user performs a current contraction, the system can measure the contraction pattern using the EMG sensors and determine current feature data for the contraction. The feature data may be time dependent and vary as the muscle activity of the user varies during the user's contraction. The current feature data can be displayed on a plot with the calibrated feature data. For example, if the calibrated feature data is displayed on a three-dimensional plot and according to three component dimensions determined to be the classification model, the current feature data can be displayed using three component dimensions on the three-dimensional plot.

In some cases, the current feature data can be dynamically displayed using a moving cursor and a trace that is based on the time varying changes of the measured EMG data over a duration of muscle contraction. Accordingly, the control signal viewer can display how the feature data from a user's contraction changes over time in the classification space and with respect to the calibrated data sets. Put another way, a user can perform a contraction and the control signal viewer can display the feature data associated with the contraction in real-time so that a user can see how their contraction relates to the calibrated feature data sets associated with different movement classifications. In this manner, a user may be able to practice particular contractions and the control signal viewer can display how those contractions are being classified in real time. In some cases, as a user performs a contraction, the control signal viewer may determine that the user's contraction would be classified as a particular movement and highlight or otherwise change how the calibrated feature data associated with that movement is displayed to indicate the current movement classification that would be used to control a prosthetic device. In this way, a user can visualize if a current contraction is being classified as the intended movement.

The control signal viewer can also provide one or more controls for updating feature data associated with a current movement classification and/or adding a new movement classification. The control signal viewer may provide an option for a user to record or otherwise collect additional EMG data that can be added to the classification model. In response to a user selecting the option to record additional data, the system may instruct a user to perform a muscle contraction, which can include instructing a user to perform a contraction for a particular movement (e.g., pinch, palm up, etc.) or perform a contraction that is not associated with a movement. The system can collect EMG data from the sensors and generate a feature data set based on the collected EMG data.

In some cases, the control signal viewer may present one or more options for using the new feature data set based on the relation of the new feature data to the calibrated feature data. For example, if the new feature data overlaps or is not well separated from a calibrated feature data set, the control signal viewer may present an option to replace or supplement the calibrated feature data with the new feature data. In other cases, if the new feature data is separated from the calibrated feature data sets, the control system may present an option to add a new movement classification to the classifier model.

In this manner, more generally and broadly, the systems and methods described herein relate to adaptive and training techniques that provide robust signal classification and differentiation (e.g., myoelectric signal classification and differentiation, sonomyography signals classification and differentiation) to determine an intended user movement or pose for a prosthetic device. The viewing techniques may allow a user to visualize how the control system is classifying muscle contractions and allow a user to change the currently calibrated data to improve the performance of a user's control over a prosthetic device, such as an electromechanical prosthetic hand. In addition, the systems and methods described herein may result in reduced rejection rates and reduced instances of discontinued prosthetic use.

For simplicity of description, the embodiments described herein reference externally worn prosthetics (which may also be referred to as exoprostheses), but it may be appreciated that this is merely one example of a myoelectric prosthetic that can leverage the systems and methods described herein. For example, some embodiments described herein can apply equivalently to endoprostheses, implanted biometric devices, non-prosthetic human-machine interface devices, and so on. Further, for simplicity of description, many embodiments that follow reference a prosthetic configured for use by a user with an upper limb amputation, such as a transhumeral amputation or a transradial amputation. However, it may be appreciated that these are merely examples and other prosthetic devices can leverage the systems and methods described herein; these examples are not exhaustive.

Generally and broadly, as noted above, a user may provide instructions to a human-machine interface device (e.g., a myoelectric prosthetic device) by activating skeletal muscles and/or nerves in a residual limb and/or surrounding areas or other locations (e.g., chest, back, and so on). Activating skeletal muscle and/or nerves can generate electrical signals that can be detected by one or more electrodes (also referred to herein as “sensors,” “myoelectric sensors” or “EMG sensors”) positioned on the user's skin and/or implanted within the user's musculature. Additionally or alternatively, activated skeletal muscle and/or nerves can generate muscle activity (e.g., spatial movement) that can be detected using ultrasonic techniques (also referred to herein as “sensors,” or “sonomyographic sensors”) positioned on a user and/or implanted at least partially within the user's body. A user can generate different patterns of volitional motor control signals (e.g., myoelectric signals, sonomyography signals, and/or other suitable biological signals) that can be associated to specific movements or poses of the myoelectric prosthetic.

A calibration/training procedure can be performed to correlate different myoelectric patterns generated by a user with specific movements or poses that the prosthetic device can perform or transition to. The calibration procedure can be leveraged to train a classification model, which in turn can be used to control operation of the prosthetic device.

For example, when a user intends their prosthetic to perform a specific action, the user can cause to be generated a unique electric pattern which can be sampled by a set or array of electrodes/sensors. Output from the sensors can be provided as input to the classification model. The classification model can provide, as output, a movement or pose associated with the input signal. The pose or movement can thereafter be used by an electromechanical control system of the prosthetic to change an angular and/or linear position of one or more electromechanical actuators of the prosthetic, thereby moving the prosthetic and/or changing a pose thereof.

Traditional myoelectric controlled prosthetics are calibrated by attaching myoelectric sensors to a user and requiring the user to perform a series of tasks, which in turn are used to build a static classification model. However, once the calibration is performed, the user may not receive feedback or other information as to how a particular contraction is being classified. Accordingly, it can be difficult for a user to determine why the prosthetic device is performing unintended movements and/or for a user to train their contractions to perform a desired movement due to a lack of feedback.

By contrast, the systems and methods described herein include a control signal viewer that can display muscle contraction data in real-time using visual feedback provided to a user. In particular, in some embodiments, a user can practice particular muscle contractions which may allow a user more repeatably and/or reliably to perform muscle contractions that result in an intended classification and movement of the prosthetic device. Additionally or alternatively, a user can update feature data associated with problematic movement classifications (e.g., movement classifications with lower separation) that result in higher levels of unintended movements of the prosthetic device.

As one non-limiting example, the systems and methods described herein may be applied to a myoelectric upper limb prosthetic that includes an electromechanical hand. The system can include an array of sensors that are each positioned at a different location on a user's residual limb or supporting musculature (e.g., chest, back, neck, and so on). The array of sensors can detect volitional motor control signals (e.g., myoelectric signals, sonomyography signals, and/or other suitable biological signals) that are generated by a user when the user wants the lower limb prosthetic to perform a particular movement or action by the prosthetic. The system can generate a dataset from the volitional motor control signals (e.g., myoelectric signals, sonomyography signals, and/or other suitable biological signals) and provide the dataset as input to the classification model. The output of the classification model can be used to identify an intended user movement and the control system can send control signals to the electromechanical hand to perform the movement identified by the classification model.

The control system viewer may also be used as a training application without the need of a prosthetic device such as an electromechanical hand. For example, the system can include an array of sensors that are attached to the user's residual limb and the signals obtained from the sensors can be used to perform various movement calibrations as described herein. Additionally, the control system viewer can display the calibrated data along with real-time contraction data for a user, which may allow a user to optimize the calibration feature data and/or practice contractions while receiving real-time visual feedback. The control system viewer can allow these operations to be done without needing to actuate a prosthetic device, which may allow a user to train and/or program a classifier prior to receiving or with or otherwise needing to wear the actual prosthetic device.

In some cases, the control system viewer can be implemented as a virtual reality (VR), augmented reality (AR), and/or mixed reality (MR) system, which may be generally referred to a “virtual reality or VR” system. The VR system may generate a VR environment using a head mounted display. The VR environment may include simulated environment for calibrating a classification model, viewing calibrated feature sets and/or updating the classification model as described herein. For example, the VR system may generate a VR environment that displays a multi-dimensional plot of calibrated feature data along with a real-time view of current feature data determined from a user contraction. The VR environment may display a relative positioning of the feature data in three-dimensional space and within the context of the user's current environment, which may help a user view or engage with how the classification model functions.

In some cases, the VR system may display feedback or other visual cues to a user that indicate EMG patterns of a contraction and/or how particular contractions are being classified. For example, the VR system may overlay image data onto the array of sensors that indicates how much EMG activity each sensors is currently detecting. Accordingly, the user may receive visual ques at the sensor site that indicates a current contraction pattern. Additional or alternatively, the feature data can be concurrently displayed in the VR environment, which can create a direct visual correlation between a current contraction pattern and how that contraction is being classified, which is represented in the component space. In other cases, the VR system can be used to coach and/or provide suggestions for to help a user perform contractions that result in a desired movement classification. For example, the system display a target contraction pattern, for example over the sensor array, that would result in a feature data set that would reduce misclassifications. Accordingly, a user may try to emulate the displayed contraction pattern. These are just some examples of how aspects of the present disclosure can be integrated into a VR environment.

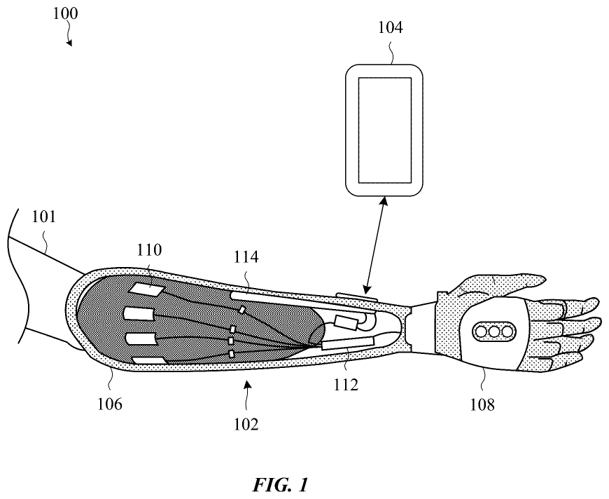

These and other embodiments are discussed below with reference to FIGS. 1-7. However, those skilled in the art will readily appreciate that the detailed description given herein with respect to these Figures is for explanatory purposes only and should not be construed as limiting.

FIG. 1 shows an example of a prosthetic system 100 (which may also be referred herein as a “human-machine interface”) including a prosthetic device 102 and a user device 104 that displays a user interface generated by the signal control viewer. The prosthetic device 102 can include a frame 106 that fits over a residual limb 101 of a user. The frame 106 can couple to an electromechanical device 108, such as a prosthetic hand. In some cases, the electromechanical device 108 can include components that control movement and/or other functions of the device. For example, the electromechanical device 108 can include motors, sensors, a control system that controls movement of device, and so on. In some cases, the electromechanical device 108 can include an independent power source. In other cases, the electromechanical device 108 can use a power source (e.g., power source 114) positioned on the frame 106.

The prosthetic device 102 can include one or more sensors 110 (one of which is labeled for clarity), a control system 112, and a power source 114. The control system 112 can exchange control commands and/or other data with the electromechanical device 108. For example, in cases where the electromechanical device 108 is a prosthetic hand, the control system 112 may send commends to the prosthetic hand that instruct the prosthetic hands to perform a specific movement, such as opening the fingers of the hand. The prosthetic hand may receive the command and one or more onboard processors can cause the hand to perform the requested function(s).

The sensors 110 can be configured to detect volitional motor control signals (e.g., myoelectric signals, sonomyography signals, and/or other suitable biological signals) at the user's residual limb 101. The sensors 110 can include multiple electrodes that couple to the frame 106 and contact the user's residual limb 101 when the prosthetic device 102 is worn by the user. Additionally or alternatively, the sensors 110 can include ultrasonic sensor(s) that coupled to the frame 106 and contact the user's residual limb 101 when the prosthetic device is worn by the user. The sensors 110 can be communicably coupled to the control system 112.

The power source 114 can be coupled to the frame 106, and in some cases, may be positioned on an inside portion of the frame. The power source 114 may be a flexible power source that can conform to an interior surface of the frame 106 and/or a portion of the user's residual limb 101. The power source 114 can include a battery or other suitable power source as described herein and may be removed and/or recharged.

In some cases, the frame 106 can include a coupling mechanism that allows different electromechanical devices 108 to be removably coupled to the frame 106. Additionally, the coupling mechanism can allow other types of electromechanical devices to be coupled to the frame and/or the frame 106 can include different types of coupling mechanisms that couple with other electromechanical devices. Accordingly, the frame 106, the sensors 110, the control system 112, and the power source 114 may form a sub-prosthetic that can interface with different types of actuating protheses. This sub-prothesis may primarily function to detect a user's volitional motor control signals (e.g., myoelectric signals, sonomyography signals, and/or other suitable biological signals), process those signals to determine an intended movement, and send control commands to an electromechanical device 108 for performing the intended movement.

The user device 104 can be any suitable electronic device that includes a display and is communicably coupled with the prosthetic device 102 including a smartphone, tablet, smartwatch, computer, or other suitable electronic devices. The user device 104 can be communicably coupled to the control system 112 via any suitable wireless transmission protocol. In yet other cases, the user device 104 can be integrated with the frame 106.

The user device 104 can be configured to output information to the user and receive inputs from the user. In some cases, the user device 104 can include a touch-sensitive display, audio, haptic, and/or other output mechanism. The user device 104 may also receive a variety of input types from a user such as touch inputs (e.g., to the touch-sensitive display), speech inputs such as voice commands, and/or other suitable input types. The user device 104 can transmit indications of received user inputs to the control system 112. Accordingly, a user may use the user device 104 to visualize feature data or other control signals generated by the prosthetic device 102 and/or to provide feedback to the prosthetic device 102.

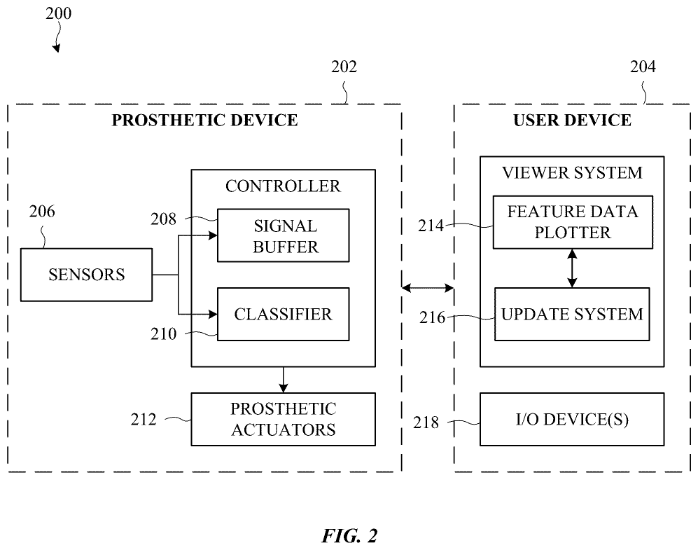

FIG. 2 shows an example process diagram 200 for adaptive control of a prosthetic device. The process flow 200 can include steps performed by a prosthetic device 202, a user device 204, and communications between these devices. The prosthetic device 202 can be an example of the prosthetic devices described herein and the user device 204 can be an example of the user devices described herein.

The prosthetic device 202 can include one or more sensors 206 (e.g., electrodes, ultrasonic sensors, and/or other suitable sensors) which can be examples of the volitional motor control sensors described herein. For example, the sensors 206 may sense myoelectric signals (and/or other muscle activity) of the user's residual limb and output data indicative of the measured myoelectric activity, sonomyographic activity, and/or other sensed signals to a signal buffer 208, which may store the data (e.g., myoelectric data, sonomyography data) for a period of time. In some cases, the data may be stored until the classifier has processed the data, the prosthetic actuators 212 have performed a movement, and the user has had an opportunity to provide feedback, for example, by recording additional data using the control signal viewer. The sensors 206 may also output the myoelectric data to the classifier 210.

The classifier 210 can include a discriminant classifier (e.g., an LDA based classifier) that takes detected myoelectric signal data as input and outputs one or more movement classifications and a confidence associated with each classification. The one or more classifications can correspond to a movement that the user intends the prosthetic device to perform. Accordingly, in some cases, the control system can output control signals that cause a prosthetic device to perform a specific movement based on the outputs from the classifier.

The classifier 210 may be calibrated using EMG data, sonomyography data, and/or other suitable data and supervised learning techniques. In some cases, the calibration data can include calibrated feature data sets that correspond to different user movements that have been trained by the classifier 210. The data set structure may allow training data for individual feature data sets to be updated independent of other feature data sets. Additionally or alternatively, the classifier 210 (e.g., LDA) may access individual data sets when classifying myoelectric data, which may increase the speed and efficiency of performing the classification functions as compared to accessing the entire movement library for all trained movements.

The output of the classifier 210 can be used to send control commands to the prosthetic actuators 212. The prosthetic actuators 212 can include the components in a prothesis that generate motion such as hand motion(s) instructed by control commands from the controller. In some cases, the control commands can cause the prosthetic to perform a movement such as opening the hand, closing the hand, or moving the hand to a specific position. In other cases, the control commands can include instructions for performing more complex operations such as a sequence of movements. For example, the control commands can instruct a sequence of movements to pick up an object and cause the prosthetic actuators 212 to perform a sequence of movements such as first opening the hand so that it can be positioned around an object and then closing the hand to grip the object.

In some cases, the prosthetic actuators 212 can include one or more sensors, which may be used to control the timing of the movements. In the sequence of opening a hand followed by closing of a hand to pick up an object, the sensors may be used to determine when the hand is ready to be closed. For example, the prosthetic actuators 212 may perform the first motion of opening the hand and then wait for feedback from one or more sensors indicating that the hand is positioned around an object. In response to the feedback, the prosthetic actuators 212 may perform the second movement of closing the hand.

The user device 204 can be sent an indication of the movements performed by the prosthetic actuators 212. In some cases, the controller may send indications of the control commands to the user device. Additionally or alternatively, the controller can send indications of alternative movements. For example, the classifier 210 may have identified two (or more) movements with high probabilities of being the intended movement of the user. The classifier may select one of these movements as the intended movement. The controller may send an indication of the selected movement to the user device and also send an indication of the next most probable movement that was not selected by the classifier 210. In some cases, the controller may send multiple alternative movements. Additionally or alternatively, the controller can send a determined probability that each movement was the intended movement of the user.

In some cases, the prosthetic device 202 can communicate with the user device 204. For example, the classifier 210 outputs and/or prosthetic actuators 212 control signals may send feature data and/or movement control data to the user device 204, which may be used to by the viewer system to display data associated with detected data. The user device 204 can implement a viewer system (also referred to herein as a “control signal viewer”), which can display calibrated feature data and/or current feature data for a user and allow a user to update the classification model.

The viewer system can include a feature data plotter 214, which can receive data from the prosthetic device including control data that is used to operate the prosthetic actuators 212. The feature data plotter 214 can process the received data and generate a user interface that displays the feature data on the user device 204, as described herein. The viewer system can provide control that allows a user to select how and/or what data is displayed in the user interface. For example, the viewer system may allow a user to view specific sets of feature data while hiding other sets of feature data. The viewer system may include controls for manipulating a view perspective of the data and so on. The update system 216 can allow a user to obtain additional data (e.g., EMG data, sonomyography data) that can be incorporated into the classification.

In response to the user selecting a record data option, an update system 216 can trigger a data acquisition process at the prosthetic device 202. The update system 216 can use this data to retrain the classifier so that newly obtained myoelectric data is used to update or replace a current feature data set or add a new movement classification to the classifier 210, as described herein.

The user device 204 can include an I/O device(s) 218 as described herein, which can be used to output information to the user and receive feedback from the user.

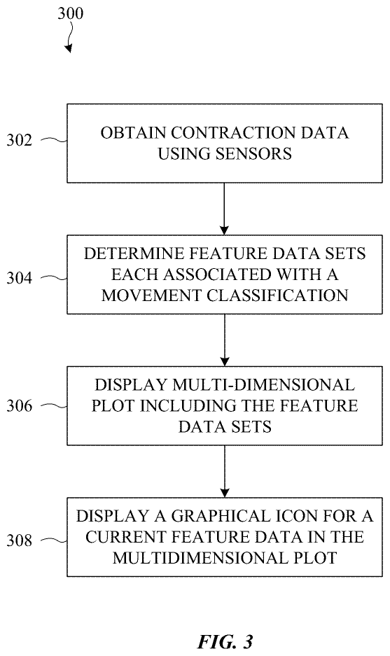

FIG. 3 shows a process 300 for generating feature data that can be displayed by the control signal viewer operating on a user device as described herein. The process 300 can be performed by a human-machine interface including the human-machine interfaces described herein. Portions of the process 300 may be performed by a prosthetic device, and other portions may be performed by a user device that is in communication with a prosthetic device.

At operation 302, the process 300 can include obtaining contraction data using sensors that contact a user. The contraction data can include signals (e.g., EMG signals, sonomyography signals) that are measured by the sensors and include measuring a contraction over a duration of the contraction or user movement. In some cases, the system can measure the contraction for a defined period of time. In other cases, the system may determine a contraction start and/or a contraction end and obtain data from the determined contraction start to the contraction end. For example, part of the calibration procedure may be determining a rest state of a user, which can correspond to EMG activity while the user is resting (e.g., not contracting) their limb and/or other portions of their body. The system may define a set of rest conditions that can include a threshold amount of EMG activity. The system may identify when current EMG activity sensed by the sensors is within the defined rest state. The system may identify the start of a contraction based on the sensed EMG activity of the user exceeding or otherwise being outside of the defined rest state. Additionally, the system may identify the end of a contraction based on when the sensed EMG activity of the user returns within the defined rest state.

In some cases, determining a rest state for a user may be performed in response to initiating a calibration procedure. The EMG data obtained during the rest state calibration can be used along with one or more data sets for different movements and can be used to configure the classification model. Obtaining contraction data for one or more movements can include instructing or otherwise prompting a user to perform a contraction for a specific movement and obtaining EMG data during the contraction. The system can prompt the user to perform multiple different movements and obtain EMG data during each of the contraction periods.

At operation 304, the process 300 can include determining one or more feature data sets that are each associated with a different movement classification. The EMG data obtained during operation 304 can be input into a classification model, such as a classification model that performs a discriminate analysis (e.g., a LDA) to separate the different EMG data. In some cases, the discriminate analysis can be configured to identify one or more component dimensions that increase a separation of data clusters between the EMG data for each movement set in the component space while reducing the scatter of each cluster. The output of the discriminate analysis can include feature data sets (e.g., clusters of data) in the determined component space that each correspond to a different movement classification. For example, a first feature data set may include a first set of data points (first data cluster) in the component space that is associated with a first movement classification (e.g., a pinch movement). A second feature data set may include a second set of data points (second data cluster) in the component space that is associated with a second movement classification (e.g., a flat hand pose).

The system can determine a feature data set for each movement that EMG data was obtained. In some cases, the feature data may include a reduced data set that includes down-sampling or performing other signal acquisition or statistical techniques such as filtering noise and so on. Additionally or alternatively, the feature data set can be obtained from a user performing a specific movement one time or multiple times. For example, at operation 302, the system may instruct the user to perform a specific movement multiple times and combine the obtained EMG data from the multiple contractions to generate a feature data set for a particular movement classification. In some cases, the process 300 can instruct a user to perform the same movement multiple times in a row and/or vary the sequence of the calibrated movements obtained, which may help emulate real world use. For example, if the process 300 includes calibrating three different movements—a pinch move, a palm up pose, and a palm down pose—the system may prompt the user to perform a first sequence of one or more pinch contractions, followed by a second sequence of one or more palm up contractions followed by a third sequence of one or more palm down contractions, and then repeat that process in the same or different order.

At operation 306, the process 300 can include displaying a plot that includes the calibrated feature data sets. In some cases, the plot can be a multi-dimensional plot that displays the feature data in the component space that was determined by the discriminant analysis. The multi-dimensional plot can be a two-dimensional plot that displays the feature data in a two-dimensional component space using the two most significant component dimensions identified by the discriminant analysis. In other cases, the multi-dimensional plot can be a three-dimensional plot that displays the feature data in a three-dimensional component space using the three most significant component dimensions identified by the discriminant analysis. In some cases, the component analysis and multi-dimensional plots can be configured to display the feature data using orthogonal component dimensions, although other component dimensions are possible. In other cases, the plot can be displayed as a one-dimensional plot according to a single component dimension or a higher-order plot, such as a four-dimensional plot.

The feature data may be displayed as data clusters in the multi-dimensional plot and the location of each data point may be defined according to the component dimensions associated with the multi-dimensional plot. For the sake of illustration, three-dimensional plots will be described herein, although as noted, other numbers of dimensions are within the scope of this disclosure. Different sets of data clusters associated with different movement classifications can be displayed differently in the multi-dimensional plot and/or labeled to indicate the movement classification that each data cluster is associated with.

At operation 308, the process 300 can include displaying a graphical icon for current feature data set in the multi-dimensional plot. Put another way, the system may display contraction data in real-time or near real-time (the “current feature data”) as it is obtained by the sensors and classified by the classifier. The system can display the current feature data with respect to the calibrated feature data sets (e.g., data clusters for each calibrated movement classification). Accordingly, a user may view how a current contraction would be classified by the system. In some cases, if the current feature data enters a classification boundary for a calibrated feature data set, the system may change an appearance of the data cluster associated with the classified movement. This may allow a user to visualize that a current contraction is being classified as a particular movement.

In some cases, the graphical icon can include a dynamic cursor or other discrete icon that moves within the multi-dimensional plot as the detected EMG pattern during a contraction change. For example, as a user performs a contraction, the EMG pattern detected by the sensors can vary with time over the duration of the contraction. The currently obtained EMG data can be classified and the graphical icon can be dynamically updated to display the changing EMG pattern of the contraction. In some cases, the graphical icon can be displayed as a trace that shows how the obtained EMG data and classified feature data changes over time. In some cases, the trace can be configured to fade over time, for example, portions of the trace that are displayed for a defined duration can be removed from the multi-dimensional plot.

Additionally or alternatively, similar processes can be performed using sonomyography signals and/or other signals that indicate muscle activity.

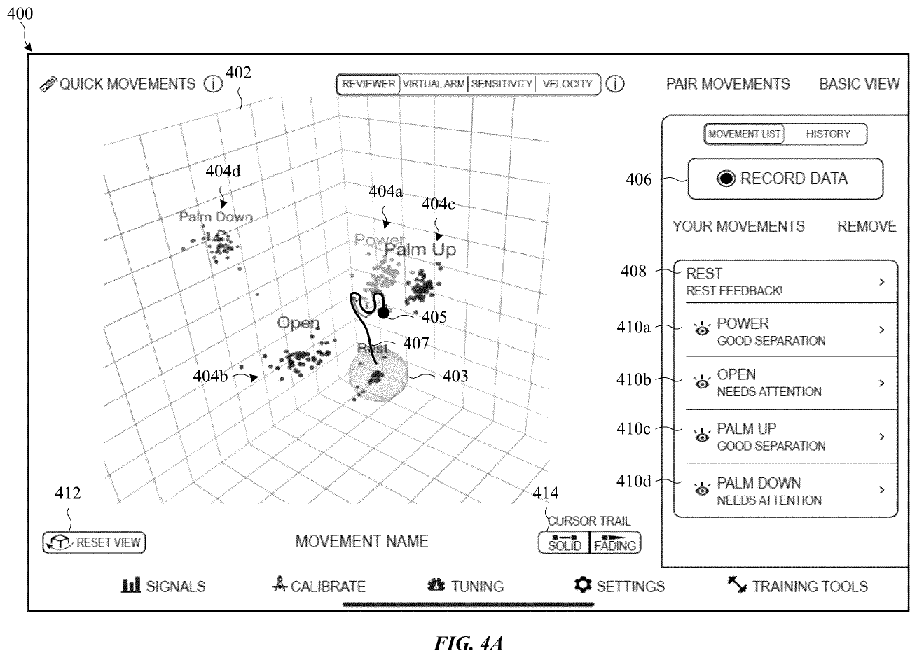

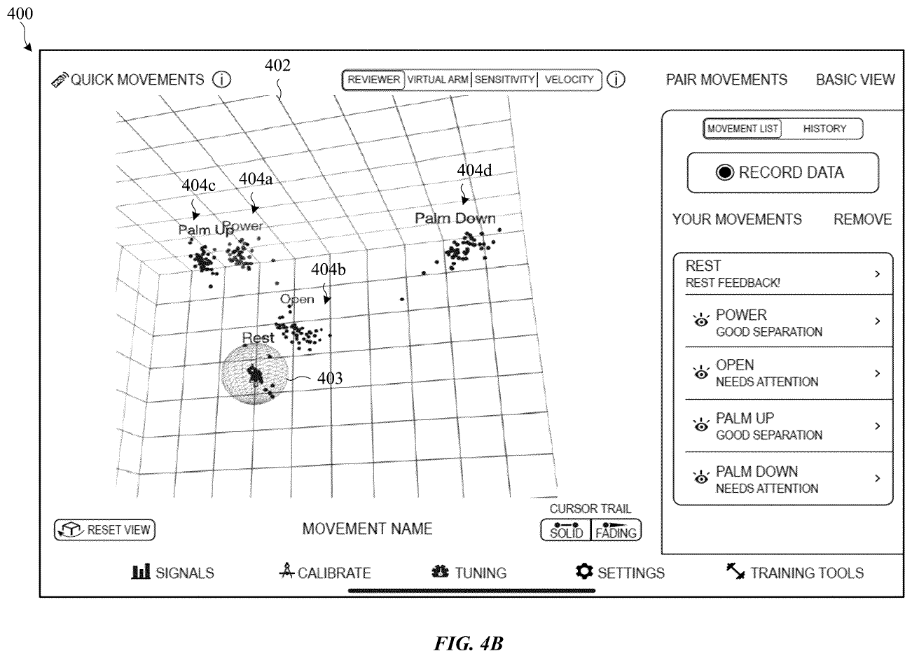

FIGS. 4A-4B show an example user interface 400 of a control signal viewer that displays feature data for a user of a prosthetic device as described herein. FIG. 4A shows the user interface 400 displaying a multi-dimensional plot 402 using a first view perspective and FIG. 4B shows the user interface 400 displaying the multi-dimensional plot 402 using a second view perspective that is different from the first view perspective. In the illustrated example, the multi-dimensional plot 402 is displayed using three component dimensions as described herein.

The multi-dimensional plot 402 can display visual elements 404 for multiple sets of feature data. In the illustrated example the visual elements 404 are displayed as point clusters that each correspond to a different movement classification as described herein. However, in other cases, the visual elements can be displayed using other techniques such as shading, a boundary envelope, or any other suitable visual indicator of the boundary for a particular movement classification. The multi-dimensional plot 402 can include a first visual element 404a (e.g., a first point cluster) that corresponds to a first movement classification (e.g., power movement), a second visual element 404b (e.g., a second point cluster) that corresponds to a second movement classification (e.g., open hand pose), a third visual element 404c (e.g., a third point cluster) that corresponds to a third movement classification (e.g., a palm up pose) and a fourth visual element 404d (e.g., a fourth point cluster) that corresponds to a fourth movement classification (e.g., a palm down pose).

The multi-dimensional plot 402 can also display a rest visual element 403 that corresponds to a rest state of the user. In some cases, the rest state and corresponding rest visual element 403 may indicate EMG activity (in the component space) that falls within the calibrated rest state. That is, even at rest (no intended contractions), the sensors may detect some EMG activity of a user and define a rest state to include some EMG activity. The rest visual element 403 and the visual elements 404 can be displayed based on their relative positions in the component state as described herein.

The rest visual element 403 and the visual elements 404 can be displayed to different classifications from each other. For example, the different visual elements 404 can be displayed using different colors, point shapes, brightness, boldness, transparency or any other suitable techniques that differentiate different visual elements 404 from each other.

The multi-dimensional plot 402 can also display a graphical icon 405 indicating a current feature data for a user. The graphical icon 405 can be dynamically updated based on changes in the EMG data over the course of a contraction, as described herein. In some cases, as the user feature data for a current contraction is determined to correspond to a classified movement, which may be displayed by the graphical icon 405 moving into a boundary (point cluster) associated with the classified movement, the visual element 404 associated with the classified movement can change appearance to indicate that the current contraction is being classified by the classifier at that particular movement. Accordingly, a user may view in real-time how a current contraction is being classified by the classifier.

The user interface 400 can also include a record option 406 that can be used to initiate a process that obtains additional EMG data and uses the data to update the classification model. For example, in response to selecting the record option 406, the system can initiate a process that measures additional EMG data and updates a current feature data set or allows a user to create a new feature data set for a new movement classification, as described herein.

In some cases, the system can be configured to determine a separability parameter for one or more of the movement classifications and indicate the separability in the user interface 400. For example, the user interface 400 can include a movement list that includes visual elements 408, 410 for each of the calibrated movements. In some cases, one or more test sets can be run through the classification model to determine a classification accuracy and the classification accuracy can be indicated by the visual elements 408, 410. For example, a first visual element 410a corresponding to the first movement classification can indicate that the feature data set for the first movement classification has good separation from the other feature data sets. The separation metric indicating how well a feature data set is separated can be determined based on a minimum amount of separation between two feature data sets and/or a determined misclassification rate (e.g., error rate) from one or more test sets. A second visual element 410b corresponding to a second movement classification can indicate that the feature data set for the second movement classification has less separation and is resulting in a higher misclassification rate with one or more other feature data sets. The user interface 400 can include a third visual element 410c indicating a separation metric for a third movement classification and a fourth visual element 410d indicating a separation metric for a fourth movement classification.

The user interface 400 can change a view perspective of the multi-dimensional plot 402, for example, from a first view perspective as shown in FIG. 4A to a second view perspective as sown in FIG. 4B. In some cases, the multi-dimensional plot 402 can dynamically change the view perspective, for example by rotating the multi-dimensional plot 402 along a defined path, which may help a user view a relative relationship of the rest visual element 403, the visual elements 404, and the graphical icon 405. For example, in the three-dimensional space one or more visual elements 404 can appear close together and/or overlapping from a first view perspective but show that the feature data sets (and visual elements 404) well separated from a different view perspective. In some cases, the system can be configured to display a view perspective based on a separation of one or more of the feature data sets (and corresponding visual elements 404). For example, the system can display the plot 402 from a view perspective that shows the minimum distance between two adjacent feature data sets.

The user interface 400 can include controls 412 and 414 that can be used by a user to manually adjust a view perspective of the plot 402. For example, a first control 412 may return the plot 402 to a default view, which may display a perspective view of the component dimensions. A second control 414 may change how the graphical icon 405 is displayed for example by showing a solid cursor trace 407 or a fading cursor trace 407 as described herein. Additionally or alternatively, the system can be configured to update the view perspective based on touch-inputs, which can be used to zoom, pan, rotate, or perform any other suitable view manipulations to the plot 402.

Additionally or alternatively, the user interface 400 of a control signal viewer can display feature data for a user of a prosthetic device based on sensing myography signals and/or other suitable biologicals signals, as described herein.

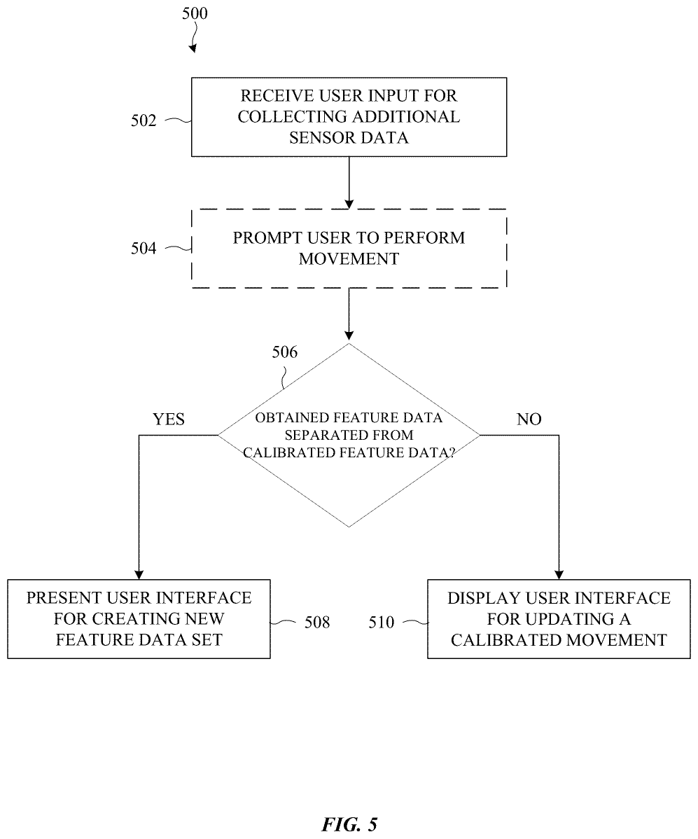

FIG. 5 shows a process 500 for updating a calibrated movement or creating a new movement based on feature data collected using the control signal viewer. The process can be performed by a human-machine interface including the human-machine interfaces described herein.

At operation 502, the process 500 can include receiving a user input for the system to collect additional sensor data. In some cases, the input can be a selection of a record new data option displayed in a user interface of the control signal viewer, as described herein. The user input can be used to initiate a data collection process at the sensors, which can obtain EMG data for a user as described herein.

At operation 504, the process 500 can prompt a user to perform a specific type of movement. In some cases, the user may indicate or select a particular movement classification to obtain new data, for example at operation 502. In response to identifying a particular movement classification, the system may prompt the user to perform one or more contractions associated with the particular movement. In some cases, the system can indicate a status of the data collection such indicating a duration for collecting the contraction data and/or indicating a status of the amount of data that is being collected (e.g., a status bar).

In some cases, EMG data can be collected without specifying a particular movement classification. Additionally or alternatively, the user may collect data related to a rest state and may not perform any contraction while obtaining additional EMG data from the sensors.

At operation 506, the process 500 can include determining a relationship of the obtained data to the calibrated sets of feature data. For example, the system can determine whether the newly obtained data is separated from one or more of the calibrated feature data sets. In some cases, this can include determining if the newly obtained data overlaps the existing feature data set. In other cases, the system may use one or more test data sets to determine a classification error rate between a new set of feature data determined from the newly obtained sensor data and a calibrated set of sensor data. If the classification error rate satisfies a defined error rate, the system may determine that the newly obtained data set is not separated from the classified data set.

If the system determines at operation 506 that the newly obtained data is separated from the calibrated feature data, at operation 508, the process 500 can include displaying a user interface for defining a new movement classification using the newly obtained data. The user may be able to define a name for the new movement class and the system can update the classifier to include the new feature data and the corresponding new movement classification.

If the system determines at operation 506 that the newly obtained data is not separated from a calibrated set of feature data, at operation 510, the process 500 can include displaying a user interface for updating a calibrated feature data set. In some cases, the user interface can display an option for replacing a calibrated feature data set with a new feature data set based on the newly obtained data. In other cases, the user interface can display an option for adding the newly obtained data to the calibrated feature data set.

Additionally or alternatively, similar processes can be performed using sonomyography signals and/or other signals that indicate muscle activity.

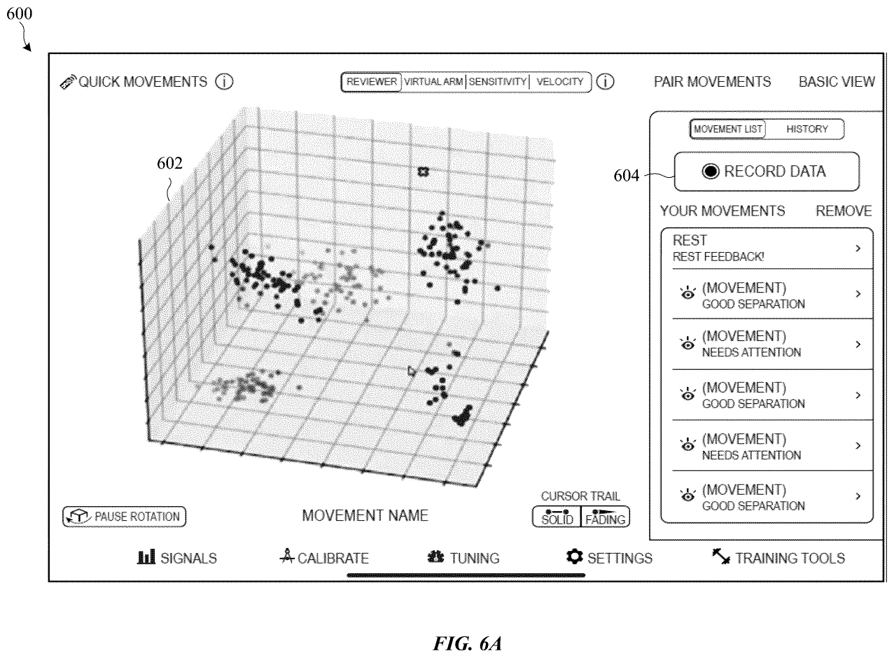

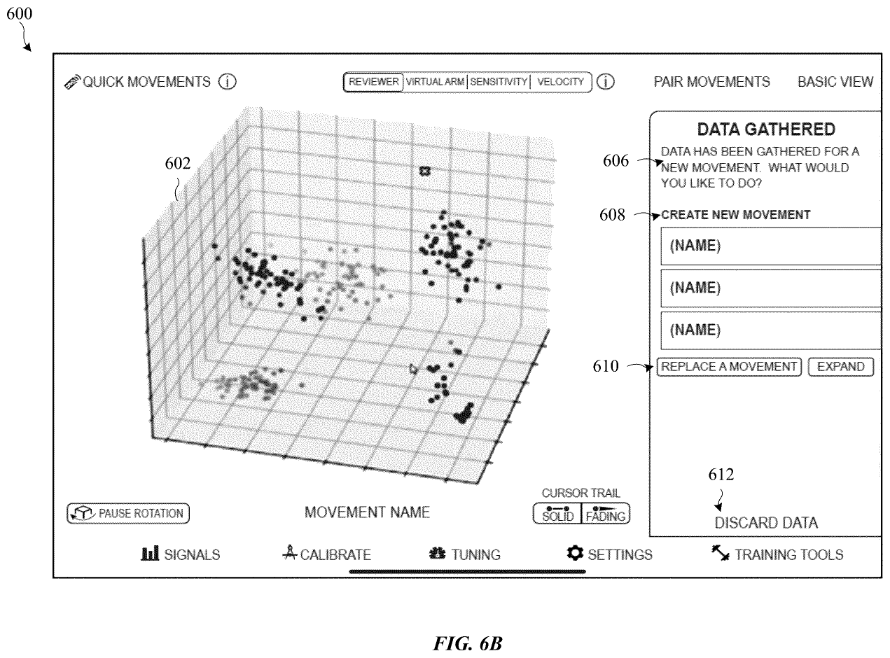

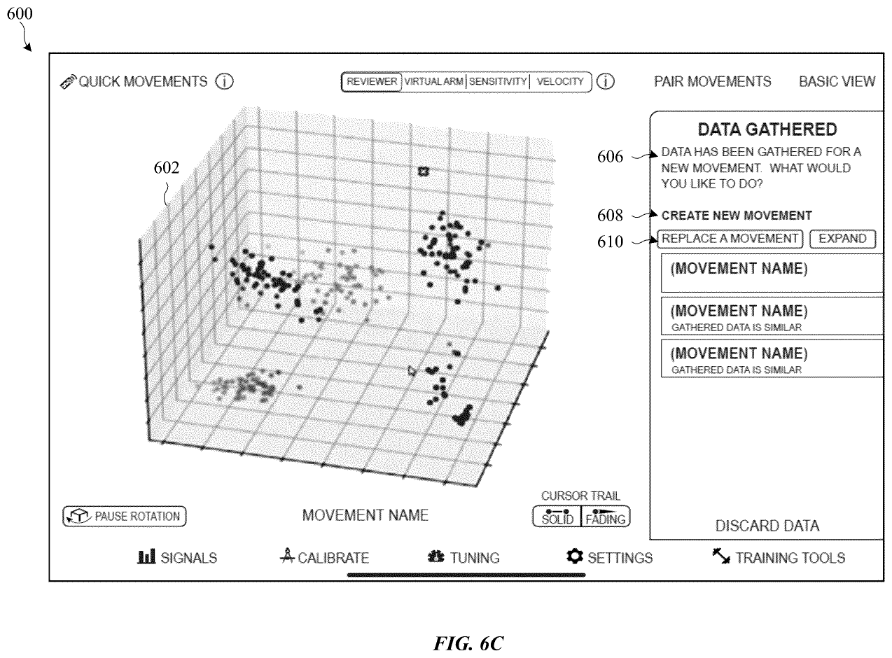

FIGS. 6A-6C show an example user interface 600 of a control signal viewer that can be used to visualize and obtain additional feature data. The user interface 600 can be generated by the control signal viewer and displayed by a human-machine interface including the human-machine interfaces described herein.

As shown in FIG. 6A the user interface 600 can include a plot 602 and an option 604 to obtain additional sensor data that can be used to update the classification model. The plot 602 can be a multi-dimensional plot that displays visual elements corresponding to feature data for the calibrated movement classifications, as described herein. In response to the user selecting the option 604 to obtain additional sensor data, the system can be configured to initiate a data collection procedure and obtain EMG data, sonomyography data and/or other suitable data from the sensors, as described herein.

In some cases, the system can determine how the newly acquired data relates to the calibrated data. For example, the system may input the newly acquired data into the calibration model to generate a new feature data set in the component space. In some cases, the control signal viewer can display a visual element for the new feature data in the plot 602, which can allow a user to visually see how the new feature data relates to the calibrated feature data. Additionally or alternatively, the system can determine whether the new feature data is separated or overwise overlaps with classification boundaries of the calibrated feature data sets. In some cases, the system determines a minimum separation between the new feature data set and the closest calibrated feature data set. If the minimum separation is below a defined metric, the system may determine that the new feature data set is not separated from the calibrated set. This may be used to identify feature data sets that are close and likely to result in higher misclassification rates but may not be overlapping. In other cases, the system can use test data to determine an error rate associated with the new feature data set and/or how the new feature data set affects the error rates of the calibrated feature data sets.

As shown in FIG. 6B, if the system determines that the new feature data set is separated from the calibrated feature data sets, the control signal viewer can cause the user interface 600 to display a create new movement tool 608, which can be used to generate a new movement classification in the classification model. In this case, the system may recalibrate the classification model using the new feature data. The create new movement tool 608 may include an option for identifying the type of movement or pose. In some cases, this can include displaying a set of movement options that can be performed by the prosthetic devices. In response to selecting a specific movement, the control signal viewer can cause the calibration model to be updated with the new movement classification and feature data. In some cases, the user interface 600 can also display a replace movement tool 610, which can be used to update a current calibrated movement. The user interface can also display a discard option 612, which can be used to discard the newly obtained data.

FIG. 6C shows an example of the user interface 600 when the system determines that the new feature data set is not separated from a calibrated future data set. In these cases, the user interface 600 may direct a user to replace movement tool 610. The control signal view may display the calibrated feature data sets that overlap with the new feature data set in the replace movement tool 610 and one or more options for updating the calibrated set of feature data. For example, in some cases, the user may replace the calibrated feature data set with the new feature data. In other cases, the user may add the new feature data to the calibrated feature data set.

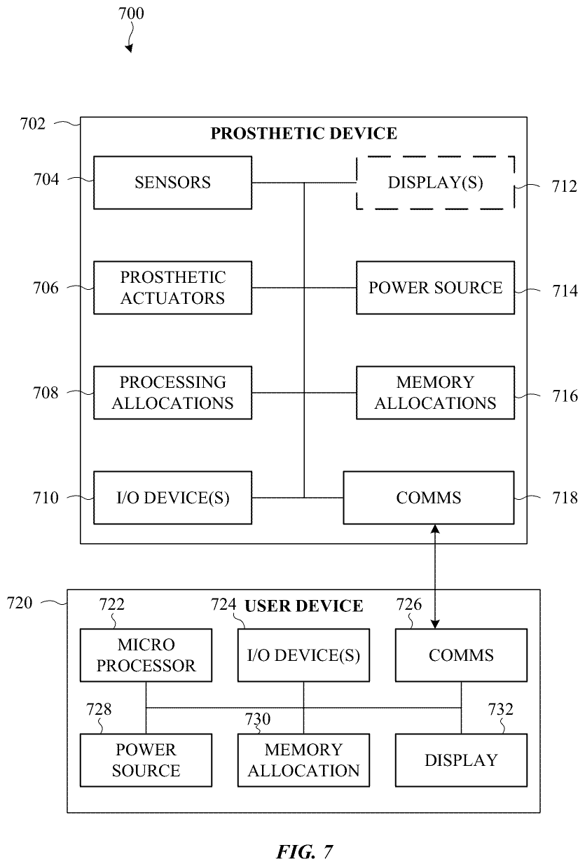

FIG. 7 shows an example electrical block diagram of a human-machine interface 700 that may perform the operations described herein. The human-machine interface 700 can have a prosthetic device 702, which can be an example of the prosthetic devices described herein. The prosthetic device 702 can include one or more sensors 704, one or more prosthetic actuators 706, processing allocations 708, input/output (I/O) devices 710, a display 712 (which may, in some examples, be optional like other components shown in FIG. 7), a power source 714, memory allocation 716, and communication devices (COMMS) 718. The human-machine interface 700 can also include a user device 720 that is an example of the user devices described herein. The user device 720 can include a micro-processor 722, I/O devices 724, communication devices (COMMS) 726, a power source 728, memory allocation 730, and a display 732.

The human-machine interface may also include one or more sensors 704 positioned at different locations on the human-machine interface 700. The sensor(s) 704 can be myoelectric sensors or other suitable sensors (e.g., sonomyographic sensors) that are configured to sense volitional motor control signals (e.g., myoelectric signals, sonomyography signals, and/or other suitable biological signals) in a residual limb of a user as described herein. Additionally, the system can include other sensors 704 configured to sense one or more types of parameters, such as but not limited to, pressure, light, touch, heat, movement, relative motion, biometric data (e.g., biological parameters), and so on.

For example, these additional sensor(s) 704 may include a thermal sensor, a position sensor, a light or optical sensor, an accelerometer, a pressure transducer, a gyroscope, a magnetometer, a health monitoring sensor, and so on. Additionally, the one or more sensors 704 can utilize any suitable sensing technology, including, but not limited to, capacitive, ultrasonic, resistive, optical, ultrasound, piezoelectric, and thermal sensing or imaging technology.

The prosthetic actuators 706 can include motors, hydraulic actuators, mechanical linkages, gear driven systems, and/or other electromechanical systems that create movement in one or more portions of the prosthetic device such as a prosthetic hand. The prosthetic actuators can include motors and assemblies that drive movement of one or more prosthetic fingers, wrist movement, other hand movement, and/or movements of other joints such as an elbow in the case of an upper arm amputation.

The processing allocations 708 can be implemented as any electronic device capable of processing, receiving, or transmitting data or instructions. For example, the processing allocations 708 can be a microprocessor, a central processing unit (CPU), an application-specific integrated circuit (ASIC), a digital signal processor (DSP), or combinations of such devices. As described herein, the term “processor” is meant to encompass a single processor or processing unit, multiple processors, multiple processing units, or other suitable computing element or elements. The processing unit can be programmed to perform the various aspects of the systems described herein.

It should be noted that the components of the human-machine interface 700 can be controlled by multiple processors. For example, select components of the human-machine interface 700 (e.g., a sensor 704) may be controlled by a first processor and other components of the human-machine interface 700 (e.g., the prosthetic actuators 706) may be controlled by a second processor, where the first and second processors may or may not be in communication with each other.

The I/O devices 710 can be any suitable mechanisms that allows a user to provide input to the prosthetic device 702 and/or the user device 720 and receive feedback from these devices. In some cases, the I/O devices 710 can be touch- and/or force-sensitive and include or be associated with touch sensors and/or force sensors that extend along the output region of the display and which may use any suitable sensing elements and/or sensing techniques.

Using touch sensors, the prosthetic device 702 and/or user device 720 may detect touch inputs applied to a display region including detecting locations of touch inputs, motions of touch inputs (e.g., the speed, direction, or other parameters of a gesture applied to the display), or the like. Using force sensors, the prosthetic device 702 and/or user device 720 may detect amounts or magnitudes of force associated with touch events applied to the display. The touch and/or force sensors may detect various types of user inputs to control or modify the operation of the device, including taps, swipes, multiple finger inputs, single- or multiple-finger touch gestures, presses, and the like.

Additionally or alternatively, the I/O devices 710 can include buttons, or other suitable input devices. In some cases, the I/O devices 710 can include a microphone and/or speaker and be configured to output sounds and receive voice-feedback. For example, a user may be able to provide voice feedback via the user device 720 on movement performed by their prosthetic device.

As noted above, the human-machine interface 700 may optionally include the display 712 such as a liquid-crystal display (LCD), an organic light emitting diode (OLED) display, a light emitting diode (LED) display, or the like. If the display 712 is an LCD, the display 712 may also include a backlight component that can be controlled to provide variable levels of display brightness. If the display 712 is an OLED or LED type display, the brightness of the display 712 may be controlled by modifying the electrical signals that are provided to display elements. The display 712 may correspond to any of the displays shown or described herein.

The power source 714 can be implemented with any device capable of providing energy to the prosthetic device 702. For example, the power source 714 may be one or more batteries or rechargeable batteries. Additionally or alternatively, the power source 714 can be a power connector or power cord that connects the prosthetic device 702 to another power source, such as a wall outlet.

The memory allocations 716 can store electronic data that can be used by the prosthetic device 702. For example, the memory 716 can store electrical data or content such as, for example, audio and video files, documents and applications, device settings and user preferences, timing signals, control signals, and data structures or databases. The memory 716 can be configured as any type of memory. By way of example only, the memory 716 can be implemented as random access memory, read-only memory, Flash memory, removable memory, other types of storage elements, or combinations of such devices.

The communication device 718 can transmit and/or receive data from a user or another electronic device. A communication device can transmit electronic signals via a communications network, such as a wireless and/or wired network connection. Examples of wireless and wired network connections include, but are not limited to, cellular, Wi-Fi, Bluetooth, IR, and Ethernet connections. In some cases, the communication device 718 can communicate with an external electronic device, such as a smartphone, electronic device, or other portable electronic device, as described here.

The user device 720 can include a micro-processor 722, I/O devices 724, communication devices 726, a power source 728, memory allocations 730, and a display 732, which can be housed in an independent structure that functions independently of the prosthetic device 702.

In other cases, the user device 720 can be configured to conductively couple to the prosthetic device 702, for example by a data and/or power cable.

The user device 720 may be any suitable electronic device including, but not limited to a cellular phone, smart watch, tablet device, laptop computer, wearable device, and so on. These examples are not limiting; the user device 720 can be any suitable electronic device.

The micro-processor 722 of the user device 720 can be configured to cooperate with the memory allocations 730 to instantiate one or more instances of software configured to perform, coordinate, or supervise one or more operations described herein. For example, in some embodiments, the user device 720 can be configured to instantiate controller software configured to execute one or more classification or retraining operations, such as described above.

In other cases, the user device 720 can be configured to instantiate an instance of software that records a timeline of events/actions performed by the prosthetic device 702. In these examples, the user can operate the user device 720 to select an action performed by the prosthetic at a known time, but not immediately in the past. For example, in some circumstances, the user may not be immediately able to signal to the prosthetic device that a particular action or pose was incorrect. In these examples, the user may leverage the user device 720 to review actions (which may be tagged with time and/or geolocation) taken by the prosthetic, so that the user can provide input indicating that one or more actions were not correct. In a simpler phrasing, in some embodiments, the user device 720 can be used to correct any historical action taken by the prosthetic device, not just actions taken immediately preceding or near-in-time with the user's input.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

1. A human-machine interface comprising:
one or more sensors configured to detect myoelectrical signals of a user; and
an electronic device configured to:
provide a set of training data as input to a classifier, the set of training data based on a first set of the detected myoelectric signals;
receive, from the classifier:
a first set of feature data associated with a first movement classification; and
a second set of feature data associated with a second movement classification;
display a user interface comprising:
a multi-dimensional plot comprising at least two-dimensions that are each based on a component dimension determined by the classifier and used to determine the first and second sets of feature data;
a first visual element corresponding to the first set of feature data and overlayed on the multi-dimensional plot; and
a second visual element corresponding to the second set of feature data and overlayed on the multi-dimensional plot;
receive a second set of detected myoelectric signals; and
display a graphical icon on the multi-dimensional plot indicating a current feature based on the second set of detected myoelectric signals.

2. The human-machine interface of claim 1, wherein:
the first visual element comprises a first point cluster comprising data points from the first set of feature data;
the second visual element comprises a second point cluster comprising data points from the second set of feature data; and
the first point cluster is displayed using graphical icons that are different from the second point cluster.

3. The human-machine interface of claim 2, wherein:
the multi-dimensional plot comprises three dimensions each corresponding to one of three component dimensions determined by the classifier:
the first point cluster displays the data points from the first set of feature data based on the three component dimensions determined by the classifier; and
the second point cluster displays the data points from the second set of feature data based on the three component dimensions determined by the classifier.

4. The human-machine interface of claim 3, wherein:
an extracted feature is determined using the three component dimensions; and
the graphical icon is displayed using the three component dimensions.

5. The human-machine interface of claim 1, wherein the graphical icon is updated as the second set of detected myoelectric signals is received from the one or more sensors.

6. The human-machine interface of claim 5, wherein:
the graphical icon comprises a trace that includes extracted feature data that was collected within a defined interval; and
in response to a duration of a displayed portion of the trace meeting or exceeding the defined interval, the electronic device removes the displayed portion of the trace from the multi-dimensional plot.

7. The human-machine interface of claim 1, wherein:
the electronic device is further configured to determine a separability parameter between the first set of feature data and the second set of feature data; and
the electronic device is configured to display an indication of the separability parameter.

8. The human-machine interface of claim 1, wherein the electronic device is configured to dynamically change a perspective of the displayed multi-dimensional plot and the first and second visual elements.

9. A human-machine interface comprising:
one or more sensors configured to detect myoelectrical signals of a user; and
an electronic device configured to:
input first training data to a classification model, the first training data based on first myoelectric signals detected by the one or more sensors;
receive, from the classification model, multiple sets of feature data each associated with a movement classification;
display a user interface comprising:
a multi-dimensional plot comprising at least two-dimensions that are based on component dimensions determined by the classification model and used to determine the multiple sets of feature data;
visual elements each corresponding to a set of feature data of the multiple sets of feature data and overlayed on the multi-dimensional plot;
receive second training data based on second myoelectric signals detected by the one or more sensors; and
update the classification model using the second training data.

10. (canceled)

11. The human-machine interface of claim 9, wherein the electronic device is configured to, in response to updating the classification model using the second training data, update the multi-dimensional plot and the visual elements displayed at the user interface.

12. The human-machine interface of claim 9, wherein the electronic device is configured to:
receive third myoelectric signals detected by the one or more sensors; and
display a graphical icon on the multi-dimensional plot indicating a current feature based on the third myoelectric signals.

13. The human-machine interface of claim 12, wherein the electronic device is configured to indicate when the current feature is associated with one of the multiple sets of feature data.

14. The human-machine interface of claim 9, wherein each of the multiple sets of feature data correspond to a different movement classification.

15. The human-machine interface of claim 14, wherein:
the first training data comprises myoelectric signals corresponding to each of the different movement classifications; and
the second training data comprises myoelectric signals corresponding to each of the different movement classifications.

16. The human-machine interface of claim 9, wherein:
the electronic device is configured to collect the first training data as part of a training procedure; and
in response to initiating the training procedure, the electronic device is configured to prompt the user to perform one or more movements; and
the first myoelectric signals are detected in response to the user performing the one or more movements.

17. The human-machine interface of claim 9, wherein
the electronic device is further configured to determine a separability parameter between a first set of feature data and a second set of feature data of the multiple sets of feature data; and
the electronic device is configured to display an indication of the separability parameter.

18. A method for operating a prosthetic device, the method comprising:
obtaining first myoelectric signals using one or more sensors positioned at a user;
inputting first data to a classification model, the first data determined from the first myoelectric signals;
receiving from the classification model one or more sets of feature data each associated with a movement classification, the one or more sets of feature data further associated with one or more component dimensions determined by the classification model;
displaying one or more visual elements each corresponding to a set of feature data on a multi-dimensional plot, the multi-dimensional plot comprising axes corresponding to the one or more component dimensions determined by the classification model;
obtaining second myoelectric signals using the one or more sensors;
inputting second data into the classification model, the second data determined from the second myoelectric signals;
in response to inputting the second data into the classification model, receiving feature data from the classification model; and
displaying a visual icon corresponding to the feature data on the multi-dimensional plot.

19. The method of claim 18, wherein:
the visual icon is displayed while obtaining the second myoelectric signals; and
a location of the visual icon within the multi-dimensional plot is updated as the feature data is received.

20. The method of claim 18, wherein:
the one or more visual elements each comprise a point cluster;
each visual element comprises a label identifying a corresponding movement classification; and
the visual icon indicates when the received feature data overlaps with a point cluster of the one or more visual elements.

21. The method of claim 20, wherein:
the one or more component dimensions comprise three component dimensions; and
the one or more visual elements are displayed using the three component dimensions.
